(19) United States
(12) Patent Application Publication
(10) Pub. No.: US 2003/0139395 A1
(43) Pub. Date: Jul. 24, 2003

(54) Combination of an adenosine A2a receptor antagonist and an antidepressant or anxiolytic

(75) Inventors: William Greenlee, Teaneck, NJ (US); John Hunter, Warren, NJ (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(73) Assignee: Schering Corporation

(21) Appl. No.: 10/241,120

(22) Filed: Sep. 11, 2002

Related U.S. Application Data
(60) Provisional application No. 60/318,696, filed on Sep. 13, 2001.

(51) Int. Cl. A61K 31/5513; A61K 31/551; A61K 31/519; A61K 31/506; A61K 31/137

(52) U.S. Cl. 514/220; 514/267; 514/252.15; 514/649; 514/651; 514/221

(57) ABSTRACT

This invention relates to a method of treating depression and anxiety-related disorders comprising administering to a mammal in need of such treatment an effective amount of a combination of an adenosine A_{2A} antagonist and an antidepressant or an anxiolytic; another aspect of the invention is a pharmaceutical composition comprising a therapeutically effective amount of a combination of an adenosine A_{2A} antagonist and an antidepressant or anxiolytic in a pharmaceutically acceptable carrier.

### CROSS REFERENCE TO RELATED APLICATIONS

This application claims the benefit of U.S. Provisional Application 60/318,696, filed Sep. 13, 2001.

### BACKGROUND

The present invention relates to a combination of an adenosine A₂ₐ receptor antagonist with an antidepressant or an anxiolytic for the treatment of depression or anxiety-related disorders. The invention also relates to pharmaceutical compositions comprising said combinations.

Adenosine is known to be an endogenous modulator of a number of physiological functions. At the cardiovascular system level, adenosine is a strong vasodilator and a cardiac depressor. On the central nervous system, adenosine induces sedative, anxiolytic and antiepileptic effects. On the respiratory system, adenosine induces bronchoconstriction. At the kidney level, it exerts a biphasic action, inducing vasoconstriction at low concentrations and vasodilation at high doses. Adenosine acts as a lipolysis inhibitor on fat cells and as an antiaggregant on platelets.

Adenosine action is mediated by the interaction with different membrane specific receptors which belong to the family of receptors coupled with G proteins. Biochemical and pharmacological studies, together with advances in molecular biology, have allowed the identification of at least four subtypes of adenosine receptors: A₁, A₂ₐ, A_{2b} and A₃. Agonist activation of A₁ and A₃ receptors is associated with inhibiting the activity of the enzyme adenylate cyclase, whereas activation of A₂ₐ and A_{2b} receptors is associated with stimulating the activity of the same enzyme. Analogs of adenosine able to interact as antagonists with the A₁, A₂ₐ, A_{2b} and A₃ receptors have also been identified.

Selective antagonists for the A₂ₐ receptor are of pharmacological interest because of their reduced level of side effects. In the central nervous system, A₂ₐ antagonists can have antidepressant properties and stimulate cognitive functions. Moreover, data has shown that A₂ₐ receptors are present in high density in the basal ganglia, known to be important in the control of movement and emotion. Hence, A₂ₐ antagonists can improve motor impairment due to neurodegenerative diseases such as Parkinson's disease, senile dementia as in Alzheimer's disease, and psychoses of organic origin.

### SUMMARY OF THE INVENTION

This invention relates to a method of treating depression or anxiety-related disorders comprising administering to a mammal in need of such treatment an effective amount of a combination of an adenosine A_{2A} antagonist and an antidepressant or an anxiolytic. In other words, the invention relates to the use of a combination of an adenosine A_{2A} antagonist and an antidepressant or an anxiolytic to treat depression or anxiety-related disorders, or to the use of a combination of an adenosine A_{2A} antagonist and an antidepressant or an anxiolytic for the preparation of a medicament for the treatment of depression or anxiety-related disorders

Another aspect of the invention is a pharmaceutical composition comprising a therapeutically effective amount of a combination of an adenosine A_{2A} antagonist and an antidepressant in a pharmaceutically acceptable carrier, or a combination of an adenosine A_{2A} antagonist and an anxiolytic in a pharmaceutically acceptable carrier. Alternatively, a pharmaceutical composition comprising an adenosine A_{2A} antagonist and a separate pharmaceutical composition comprising an antidepressant or an anxiolytic can also be administered, simultaneously or sequentially, wherein the adenosine A_{2A} antagonist and the antidepressant or anxiolytic are administered in amounts chosen so that the combination is effective to treat depression or anxiety-related disorders. Kits comprising separate adenosine A_{2A} antagonist and antidepressant or anxiolytic pharmaceutical compositions in a single package are also contemplated.

### DETAILED DESCRIPTION

In the present invention, it has been discovered that compounds having adenosine A₂ₐ receptor antagonist activity, in combination with antidepressants or anxiolytic agents, are useful in the treatment of depression and anxiety-related disorders. Examples of anxiety-related disorders include social phobias, panic attack, generalized anxiety disorder (GAD), obsessive-compulsive disorders (OCD), and post-traumatic stress disorder (PTSD). The combination of the invention is useful in the treatment of comorbid anxiety and depression in Parkinson's disease.

Suitable adenosine A₂ₐ receptor antagonists can be identified by the binding assay described below. Specific examples of suitable adenosine A₂ₐ antagonists include the compounds disclosed in several US patents and US and PCT patent applications.

U.S. Ser. No. 09/865,071, filed May 24, 2001, equivalent to WO 01/92264, discloses compounds having the structural formula I

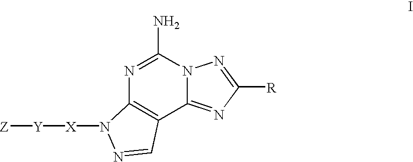

or a pharmaceutically acceptable salt thereof, wherein

R is R¹-furanyl, R¹-thienyl, R¹-pyridyl, R¹-pyridyl N-oxide, R¹-oxazolyl, R¹⁰-phenyl, R¹-pyrrolyl or C₄-C₆ cycloalkenyl;

X is C₂-C₆ alkylene or —C(O)CH₂—;

Y is —N(R²)CH₂CH₂N(R³)—, —OCH₂CH₂N(R²)—, —O—, —S—, —CH₂S—, —(CH₂)₂—NH—, or

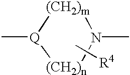

and

Z is R⁵-phenyl, R⁵-phenyl(C₁-C₆)alkyl, R⁵-heteroaryl, diphenylmethyl, R⁶—C(O)—, R⁶—SO₂—, R⁶—OC(O)—, R⁷—N(R⁸)—C(O)—, R⁷—N(R⁸)—C(S)—,

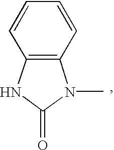

phenyl-CH(OH)—, or phenyl-C(═NOR²)—; or when Q is

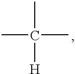

Z is also phenylamino or pyridylamino; or

Z and Y together are

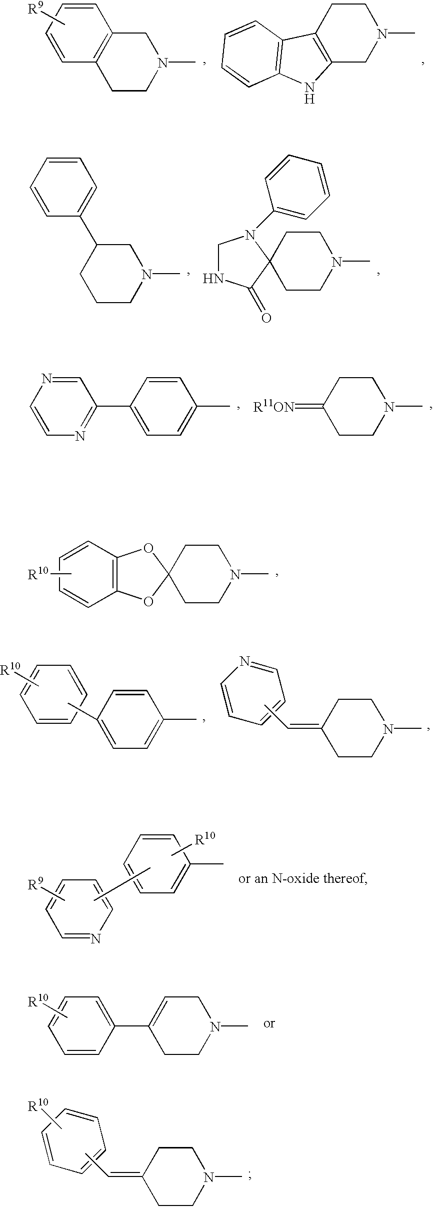

R¹ is 1 to 3 substituents independently selected from hydrogen, C₁-C₆-alkyl, —CF₃, halogen, —NO₂, —NR¹²R¹³, C₁-C₆ alkoxy, C₁-C₆ alkylthio, C₁-C₆ alkylsulfinyl, and C₁-C₆ alkylsulfonyl;

R² and R³ are independently selected from the group consisting of hydrogen and C₁-C₆ alkyl;

m and n are independently 2-3;

Q is

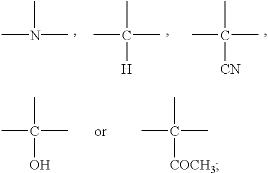

R⁴ is 1-2 substituents independently selected from the group consisting of hydrogen and C₁-C₆alkyl, or two R⁴ substituents on the same carbon can form ═O;

R⁵ is 1 to 5 substituents independently selected from the group consisting of hydrogen, halogen, C₁-C₆ alkyl, hydroxy, C₁-C₆ alkoxy, —CN, di-((C₁-C₆)alkyl)amino, —CF₃, —OCF₃, acetyl, —NO₂, hydroxy(C₁-C₆)alkoxy, (C₁-C₆)-alkoxy(C₁-C₆)alkoxy, di-((C₁-C₆)-alkoxy)(C₁-C₆)alkoxy, (C₁-C₆)-alkoxy(C₁-C₆)alkoxy-(C₁-C₆)-alkoxy, carboxy(C₁-C₆)-alkoxy, (C₁-C₆)-alkoxycarbonyl(C₁-C₆)alkoxy, (C₃-C₆)cycloalkyl(C₁-C₆)alkoxy, di-((C₁-C₆)alkyl)amino(C₁-C₆)alkoxy, morpholinyl, (C₁-C₆)alkyl-SO₂—, (C₁-C₆)alkyl-SO—(C₁-C₆)alkoxy, tetrahydropyranyloxy, (C₁-C₆)alkylcarbonyl(C₁-C₆)-alkoxy, (C₁-C₆)-alkoxycarbonyl, (C₁-C₆)alkylcarbonyloxy(C₁-C₆)-alkoxy, —SO₂NH₂, phenoxy,

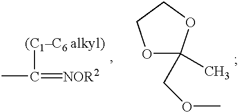

or adjacent R⁵ substituents together are —O—CH₂—O—, —O—CH₂CH₂—O—, —O—CF₂—O— or —O—CF₂CF₂—O— and form a ring with the carbon atoms to which they are attached;

R⁶ is (C₁-C₆)alkyl, R⁵-phenyl, R⁵-phenyl(C₁-C₆)alkyl, thienyl, pyridyl, (C₃-C₆)-cycloalkyl, (C₁-C₆)alkyl-OC(O)—NH—(C₁-C₆)alkyl-, di-((C₁-C₆)alkyl)aminomethyl, or

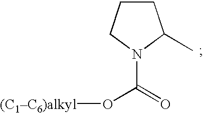

R⁷ is (C₁-C₆)alkyl, R⁵-phenyl or R⁵-phenyl(C₁-C₆)alkyl;

R⁸ is hydrogen or C₁-C₆ alkyl; or R⁷ and R⁸ together are —(CH₂)ₚ-A-(CH₂)_{q}, wherein p and q are independently 2 or 3 and A is a bond, —CH₂—, —S— or —O—, and form a ring with the nitrogen to which they are attached;

R⁹ is 1-2 groups independently selected from hydrogen, C₁-C₆ alkyl, hydroxy, C₁-C₆ alkoxy, halogen, —CF₃ and (C₁-C₆)alkoxy(C₁-C₆)alkoxy;

R¹⁰ is 1 to 5 substituents independently selected from the group consisting of hydrogen, halogen, C₁-C₆ alkyl, hydroxy, C₁-C₆ alkoxy, —CN, —NH₂, C₁-C₆alkylamino, di-((C₁-C₆)alkyl)amino, —CF₃, —OCF₃ and —S(O)₀₋₂(C₁-C₆)alkyl;

R¹¹ is H, C₁-C₆ alkyl, phenyl, benzyl, C₂-C₆ alkenyl, C₁-C₆ alkoxy(C₁-C₆)alkyl, di-((C₁-C₆)alkyl)amino(C₁-C₆)alkyl, pyrrolidinyl(C₁-C₆)alkyl or piperidino(C₁-C₆)alkyl;

R¹² is H or C₁-C₆ alkyl; and

R¹³ is (C₁-C₆)alkyl-C(O)— or (C₁-C₆)alkyl-SO₂—.

Preferred compounds of formula I are those wherein R is R¹-furanyl, R¹-thienyl, R¹-pyrrolyl or R¹⁰-phenyl, more preferably R¹-furanyl. R¹ is preferably hydrogen or halogen. Another group of preferred compounds is that wherein X is alkylene, preferably ethylene. Y is preferably

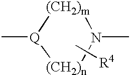

wherein Q is

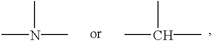

with Q preferably being nitrogen. Preferably, m and n are each 2, and R⁴ is H. A preferred definition for Z is R⁵-phenyl, R⁵-heteroaryl, R⁶—C(O)— or R⁶—SO₂—. R⁵ is preferably H, halogen, alkyl, alkoxy, hydroxyalkoxy or alkoxyalkoxy. R⁶ is preferably R⁵-phenyl.

Preferred specific compounds of formula I are those of the formula IA

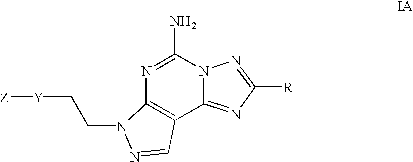

wherein R and Z-Y are as defined in the following table:

| Z—Y— | R |
|---|---|
| 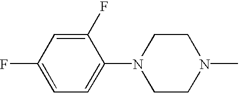 | 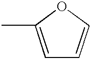 |
| 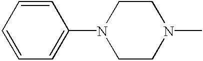 | 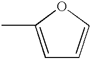 |
| 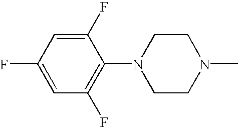 | 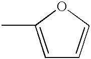 |
| 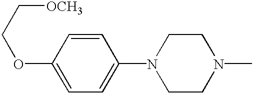 | 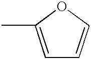 |
| 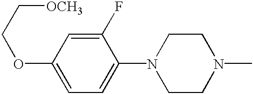 | 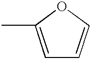 |
| 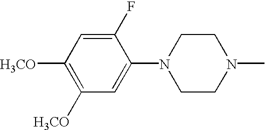 | 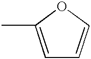 |
| 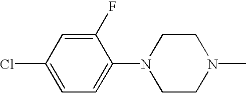 | 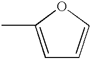 |
| 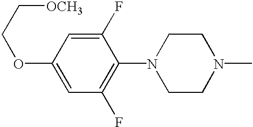 | 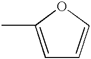 |
| 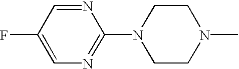 | 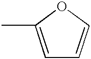 |
| 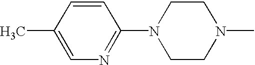 | 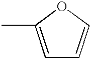 |
| 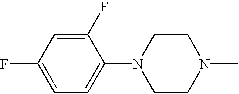 | 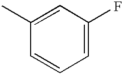 |
| 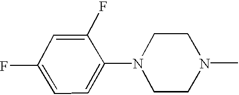 | 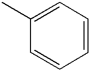 |

Other useful adenosine A₂ₐ receptor antagonists include those disclosed in WO 95/01356 as compounds having the structural formula II

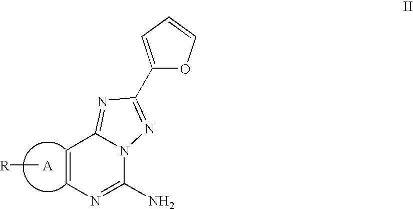

wherein:

A is pyrazole, imidazole or a triazole ring;

R is hydrogen; C₁-C₈ alkyl; C₃-C₇ alkenyl; C₃-C₇ alkynyl; C₃-C₇ cycloalkyl; C₁-C₅ alkyl substituted with one or more halogen atoms, hydroxy groups, C₁-C₄ alkoxy, C₃-C₇ cycloalkyl, groups of formula —NR₁R₂, —CONR₁R₂; aryl optionally substituted with halogen atoms, C₁-C₄ alkoxy groups, C₁-C₄ alkyl, nitro, amino, cyano, C₁-C₄ haloalkyl, C₁-C₄ haloalkoxy, carboxy, carboxyamido; C₇-C₁₀, aralkyl in which the aryl moiety can be substituted with one or more of the substituents indicated above for the aryl group; a group of formula —(CH₂)ₘ-Het, wherein Het is a 5-6 membered aromatic or non aromatic heterocyclic ring containing one or more heteroatoms selected from N, O, S and m is an integer from 1 to 5;

R₁, R₂ which are the same or different, are hydrogen, C₁-C₅ alkyl, C₇-C₁₀ aralkyl, phenyl, or taken together with the nitrogen they are linked to, form an azetidine ring or a 5-6 membered heterocyclic ring containing one or more heteroatoms such as N, O, S and n is an integer from 2 to 5.

Preferably, compounds of formula II are those wherein R is hydrogen, C₁-C₈ alkyl, aryl or C₇-C₁₀ aralkyl optionally substituted, preferably with halogen atoms.

U.S. Pat. No. 5,935,964 discloses useful adenosine A₂ₐ receptor antagonist compounds having the structural formula III

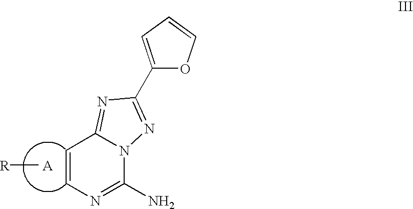

wherein

A is pyrazole, imidazole or triazole ring;

R is

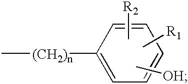

R₁ and R₂, which are the same or different, are H, OH, halogen, C₁-C₄ alkoxy, C₁-C₄ alkyl, nitro, amino, cyano, C₁-C₄ haloalkyl, C₁-C₄ haloalkoxy, carboxy or carboxamido; or the OH group, together with one of R₁ or R₂, or R₁ and R₂, can form a methylenedioxy group —O—CH₂—O—; and

n is an integer from 0-4.

Preferred compounds of formula III are those wherein A is pyrazolo[4,3-e] or 1,2,3-triazolo[5,4-e].

U.S. Pat. No. 5,565,460 discloses useful adenosine A₂ₐ receptor antagonist compounds having the structural formulas IVA and IVB, wherein formula IVA is

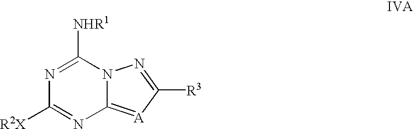

wherein

R¹ represents hydrogen, substituted or unsubstituted lower alkyl, or substituted or unsubstituted lower alkanoyl;

R² represents hydrogen, substituted or unsubstituted lower alkyl, substituted or unsubstituted lower alkenyl, substituted or unubstituted cycloalkyl, substituted or unsubstituted aryl, substituted or unsubstituted aralkyl, or a substituted or unsubstituted heterocyclic group;

R³ represents a substituted or unsubstituted heterocyclic group;

X represents a single bond, O, S, S(O), S(O)₂, or NR⁴ (in which R⁴ represents hydrogen, or substituted or unsubstituted lower alkyl; or R² and NR⁴ are combined to form a substituted or unsubstituted 4 to 6-membered saturated heterocyclic group): and

A represents N or CR⁵ (in which R⁵ represents hydrogen, or a substituted or unsubstituted lower alkyl); and

wherein formula IVB is

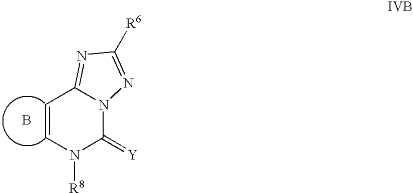

wherein

R⁶ represents substituted or unsubstituted aryl, or a substituted or unsubstituted heterocyclic group;

Y represents O, S, or NR⁷ (in which R⁷ represents substituted or unsubstituted lower alkyl, substituted or unubstituted cycloalkyl, or substituted or unsubstituted aryl);

R⁸ represents hydrogen, substituted or unsubstituted lower alkyl, substituted or unsubstituted lower alkenyl, substituted or unsubstituted lower alkynyl, substituted or unubstituted cycloalkyl, substituted or unsubstituted aryl, substituted or unsubstituted aralkyl, or a substituted or unsubstituted heterocyclic group; and

B and the adjacent two carbon atoms are combined to form a substituted or unsubstituted, partially saturated or unsaturated, monocyclic or bicyclic, carbocyclic or heterocyclic group.

U.S. Provisional Application 60/329,567 discloses useful adenosine A₂ₐ receptor antagonist compounds having the structural formula V

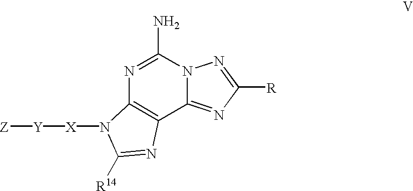

or a pharmaceutically acceptable salt thereof, wherein

R is R¹-heteroaryl, R¹⁰-phenyl, C₄-C₆ cycloalkenyl, —C(═CH₂)CH₃, —C≡C—CH₃, —CH═C(CH₃)₂,

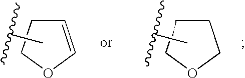

X is C₁-C₆ alkylene, —C(O)CH₂— or —C(O)N(R²)CH₂—;

Y is —N(R²)CH₂CH₂N(R³)—, —OCH₂CH₂N(R²)—, —O—, —S—, —CH₂S—, —(CH₂)₂₋₃—N(R²)—, R⁵-divalent heteroaryl, and

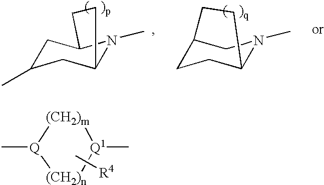

Z is R⁵-phenyl, R⁵-phenyl(C₁-C₆)alkyl, R⁵-heteroaryl, R⁵-bicyclic heteroaryl, R⁵-benzofused heteroaryl, diphenylmethyl or R⁶—C(O)—;

or when Y is

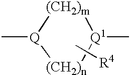

Z is also R⁶—SO₂—, R⁷—N(R⁸)—C(O)— or R⁷—N(R⁸)—C(S)—;

or when Q is

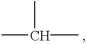

Z is also phenylamino or pyridylamino;

or Z and Y together are

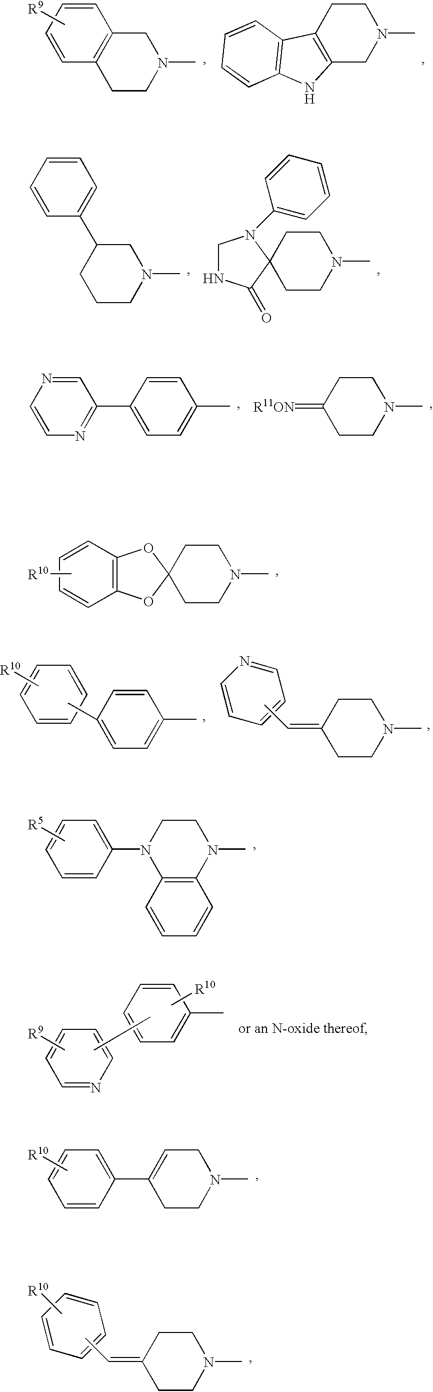

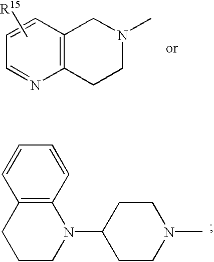

R¹ is 1 to 3 substituents independently selected from hydrogen, C₁-C₆-alkyl, —CF₃, halogen, —NO₂, —NR¹²R¹³, C₁-C₆ alkoxy, C₁-C₆ alkylthio, C₁-C₆ alkylsulfinyl, C₁-C₆ alkylsulfonyl, —COOR⁷ or —C(O)NR²R³;

R² and R³ are independently selected from the group consisting of hydrogen and C₁-C₆ alkyl;

m and n are independently 2-3;

p and q are independently 0-2;

Q and Q¹ are independently selected from the group consisting of

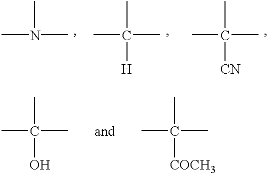

provided that one of Q and Q¹ is

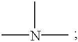

R⁴ is 1-2 substituents independently selected from the group consisting of hydrogen, C₁-C₆alkyl, R¹-aryl and R¹-heteroaryl, or two R⁴ substituents on the same carbon can form ═O;

R⁵ is 1 to 5 substituents independently selected from the group consisting of hydrogen, halogen, C₁-C₆ alkyl, hydroxy, C₁-C₆ alkoxy, —CN, di-((C₁-C₆)alkyl)amino, —CF₃, —OCF₃, acetyl, —NO₂, hydroxy(C₁-C₆)alkoxy, (C₁-C₆)-alkoxy(C₁-C₆)alkoxy, di-((C₁-C₆)-alkoxy)(C₁-C₆)alkoxy, (C₁-C₆)-alkoxy(C₁-C₆)alkoxy-(C₁-C₆)-alkoxy, carboxy(C₁-C₆)-alkoxy, (C₁-C₆)-alkoxycarbonyl(C₁-C₆)alkoxy, (C₃-C₆)cycloalkyl(C₁-C₆)alkoxy, di-((C₁-C₆)alkyl)amino(C₁-C₆)alkoxy, morpholinyl, (C₁-C₆)alkyl-SO₂—, (C₁-C₆)alkyl-SO₂—(C₁-C₆)alkoxy, tetrahydropyranyloxy, (C₁-C₆)alkylcarbonyl(C₁-C₆)-alkoxy, (C₁-C₆)-alkoxycarbonyl, (C₁-C₆)alkylcarbonyloxy(C₁-C₆)-alkoxy, —SO₂NH₂, phenoxy,

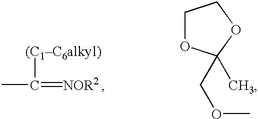

(R²O)₂—P(O)—CH₂—O— and (R²O)₂—P(O)—; or adjacent R⁵ substituents together are —O—CH₂—O—, —O—CH₂CH₂—O—, —O—CF₂—O— or —O—CF₂CF₂—O— and form a ring with the carbon atoms to which they are attached;

R⁶ is (C₁-C₆)alkyl, R⁵-phenyl, R⁵-phenyl(C₁-C₆)alkyl, thienyl, pyridyl, (C₃-C₆)-cycloalkyl, (C₁-C₆)alkyl-OC(O)—NH—(C₁-C₆)alkyl-, di-((C₁-C₆)alkyl)aminomethyl, or

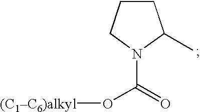

R⁷ is (C₁-C₆)alkyl, R⁵-phenyl or R⁵-phenyl(C₁-C₆)alkyl;

R⁸ is hydrogen or C₁-C₆ alkyl; or R⁷ and R⁸ together are —(CH₂)ₚ-A-(CH₂)_{q}, wherein p and q are independently 2 or 3 and A is a bond, —CH₂—, —S— or —O—, and form a ring with the nitrogen to which they are attached;

R⁹ is 1-2 substituents independently selected from the group consisting of hydrogen, C₁-C₆ alkyl, hydroxy, C₁-C₆ alkoxy, halogen, —CF₃ and (C₁-C₆)alkoxy-(C₁-C₆)alkoxy;

R¹⁰ is 1 to 5 substituents independently selected from the group consisting of hydrogen, halogen, C₁-C₆ alkyl, hydroxy, C₁-C₆ alkoxy, —CN, —NH₂, C₁-C₆alkylamino, di-((C₁-C₆)alkyl)amino, —CF₃, —OCF₃, —S(O)₀₋₂(C₁-C₆)alkyl and —CH₂—SO₂-phenyl;

R¹¹ is H, C₁-C₆ alkyl, phenyl, benzyl, C₂-C₆ alkenyl, C₁-C₆ alkoxy(C₁-C₆)alkyl, di-((C₁-C₆)alkyl)amino(C₁-C₆)alkyl, pyrrolidinyl(C₁-C₆)alkyl or piperidino(C₁-C₆)alkyl;

R¹² is H or C₁-C₆ alkyl;

R¹³ is H, (C₁-C₆)alkyl-C(O)— or (C₁-C₆)alkyl-SO₂—;

R¹⁴ is H, halogen, C₁-C₆ alkyl, hydroxy(C₁-C₆)alkyl, C₁-C₆ alkoxy(C₁-C₆)alkyl, thio(C₁-C₆)alkyl, (C₁-C₆)alkylthio(C₁-C₆)alkyl or NR²R³—(C₁-C₆)alkyl; and

R¹⁵ is H, halogen, C₁-C₆ alkyl or C₁-C₆ alkoxy.

Preferred compounds of formula V are those wherein R is R¹-furanyl, R¹-thienyl, R¹-pyrrolyl, R¹-pyridyl or R¹⁰-phenyl, more preferably R¹-furanyl or R¹⁰-phenyl. R¹ is preferably hydrogen or halogen. R¹⁰ is preferably hydrogen, halogen, alkyl or —CF₃. Another group of preferred compounds is that wherein X is alkylene, preferably ethylene. Y is preferably

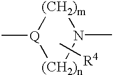

wherein Q is

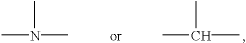

with Q preferably being nitrogen. Preferably, m and n are each 2, and R⁴ is H. A preferred definition for Z is R⁵-phenyl or R⁵-heteroaryl. R⁵ is preferably H, halogen, alkyl, alkoxy, hydroxyalkoxy or alkoxyalkoxy. R⁶ is preferably R⁵-phenyl.

Preferred specific compounds of formula V are those of the formula VA

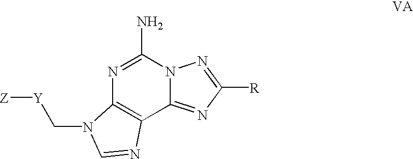

wherein R and Z-Y are as defined in the following table:

| Z—Y— | R |
|---|---|
| 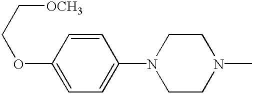 | 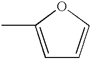 |
| 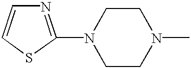 | 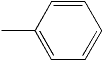 |
| 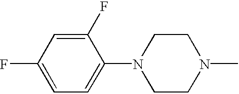 | 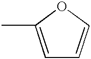 |
| 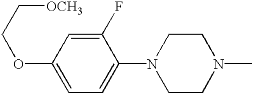 | 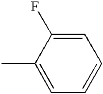 |
| 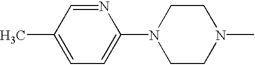 | 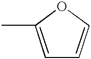 |
| 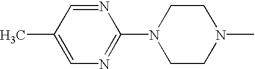 | 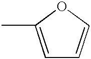 |
| 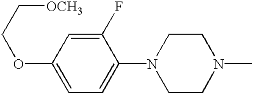 | 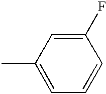 |
| 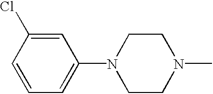 | 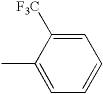 |
| 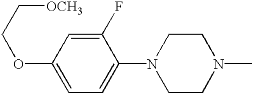 | 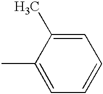 |
| 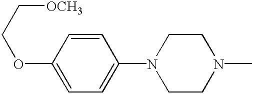 | 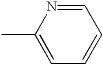 |

U.S. Provisional Application 60/334,342 discloses useful adenosine A₂ₐ receptor antagonist compounds having the structural formula VI

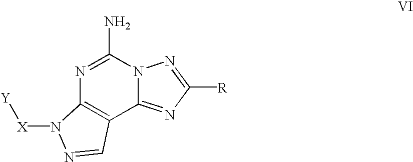

or pharmaceutically acceptable salts thereof, wherein

R is R¹-furanyl, R¹-thienyl, R¹-pyridyl, R¹-oxazolyl, R¹-pyrrolyl or R²-phenyl;

X is —(CH₂)ₙ—;

Y is a piperidinyl or pyrrolidinyl group fused to a monocyclic or bicyclic aryl or heteroaryl wherein X is attached to the N atom of the piperidinyl or pyrrolidinyl group;

n is an integer from 1 to 4;

R¹ is 1-3 substituents, which may be the same or different, and are independently selected from hydrogen, C₁-C₆-alkyl, —CF₃, halogen or NO₂; and

R² is 1-3 substituents, which may be the same or different, and are independently selected from hydrogen, C₁-C₆-alkyl, —CF₃, halogen, NO₂, C₁-C₆-alkoxy, C₁-C₆-acyloxy, C₁-C₆-alkylamino, C₁-C₆-acylamino, C₁-C₆-alkylsulfonamido, C₁-C₆-alkylaminosulfonyl, C₁-C₆-dialkylaminosulfonyl, aminosulfonyl, or hydroxyl.

In a preferred embodiment of compounds of formula VI, Y is

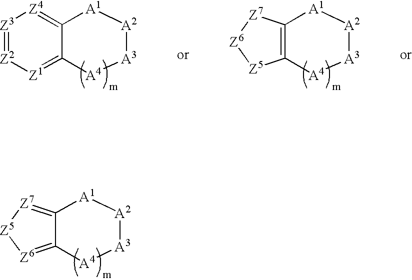

wherein A¹ is N—X, and A² and A³ each are CR⁴R⁵, or

A¹ and A³ each are CR⁴R⁵, and A² is N—X, or

A¹ and A² each are CR⁴R⁵, and A³ is N—X;

A⁴ is CR⁴R⁵;

Z¹, Z², Z³ and Z⁴ are selected from the group consisting of N and CR³, provided that 0-2 of Z¹, Z², Z³ or Z⁴ are N and the remainder are CR³;

Z⁵ is NR⁵, O, S or CR⁴R⁵;

Z⁶ is N or CR³;

Z⁷ is N or CR³;

m is an integer from 0 to 2;

R³ is hydrogen, C₁-C₆-alkyl, CF₃, halogen, NO₂, C₁-C₆-alkoxy, C₁-C₆-acyloxy, C₁-C₆-alkylamino, C₁-C₆-acylamino, C₁-C₆-alkylsulfonamino, C₁-C₆-alkylaminosulfonyl, C₁-C₆-dialkylaminosulfonyl, aminosulfonyl, or hydroxyl;

R⁴ is hydrogen, C₁-C₆-alkyl, C₁-C₆-alkoxy, —CF₃, halogen, hydroxy, or NO₂; and

R⁵ is hydrogen or C₁-C₆ alkyl.

Preferred specific examples of compounds of formula VI include compounds of the formula VIA

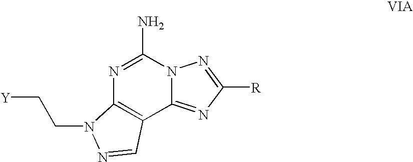

wherein Y and R are defined in the following table:

| Y | R |
|---|---|
| 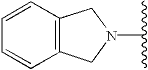 | 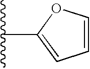 |
| 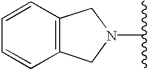 | 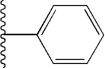 |
| 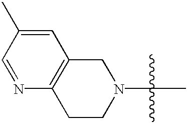 | 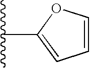 |
| 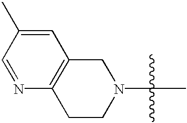 | 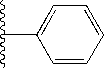 |
| 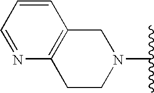 | 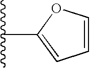 |
| 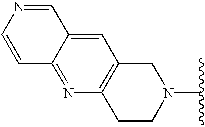 | 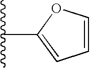 |

U.S. Provisional Application 60/334,293 discloses useful adenosine A₂ₐ receptor antagonist compounds having the structural formula VII

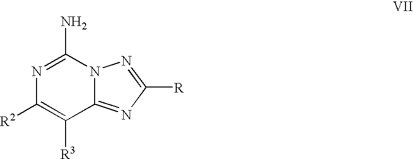

or pharmaceutically acceptable salts thereof, wherein

Q and Q¹ may be the same or different and are independently selected from the group consisting of

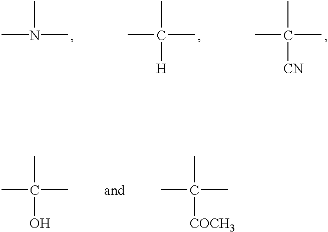

provided that one of Q and Q¹ is

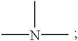

m and n are independently 1-3;

p and q are independently 0-2;

W is aryl or heteroaryl having 1-3 heteroatoms, which may be the same or different and are independently selected from N, O or S, said aryl or heteroaryl optionally substituted by 1-3 substituents, which may be the same or different and are independently selected from alkyl, halo, hydroxy, hydroxyalkyl, alkoxy, —NR⁶R⁷, (C₂-C₆)alkene, or —CN;

X is H, NH₂, —N(R⁶)(CH₂)ₘ—C₆H₅, —N(R⁶)(CH₂)ₘ₊₁—OH, —N(CH₃)₂, or

X is R¹⁸ which is attached to —Y-Z;

Y is —N(R⁶)CH₂CH₂N(R⁷)—, —OCH₂CH₂N(R⁶)—, —O—, —S—, —CH₂S—, —(CH₂)₂₋₃—N(R⁶)—, R⁸-divalent heteroaryl,

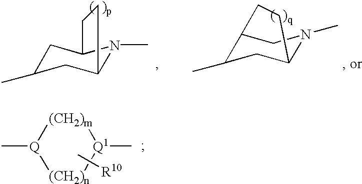

Z is alkoxyalkyl, R⁸-phenyl, R⁸-phenyl(C₁-C₆)alkyl, R⁸-heteroaryl, R⁸-bicyclic heteroaryl; R⁸-benzofused heteroaryl, diphenylmethyl or R⁹—C(O)—; or

when Y is

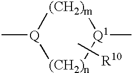

Z may also be H, R⁹—SO₂—, R¹⁷—N(R¹¹)—C(O)— or R¹⁷—N(R¹¹)—C(S)—; or

when Q is

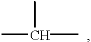

Z may also be phenylamino or pyridylamino; or

Z and Y taken together are

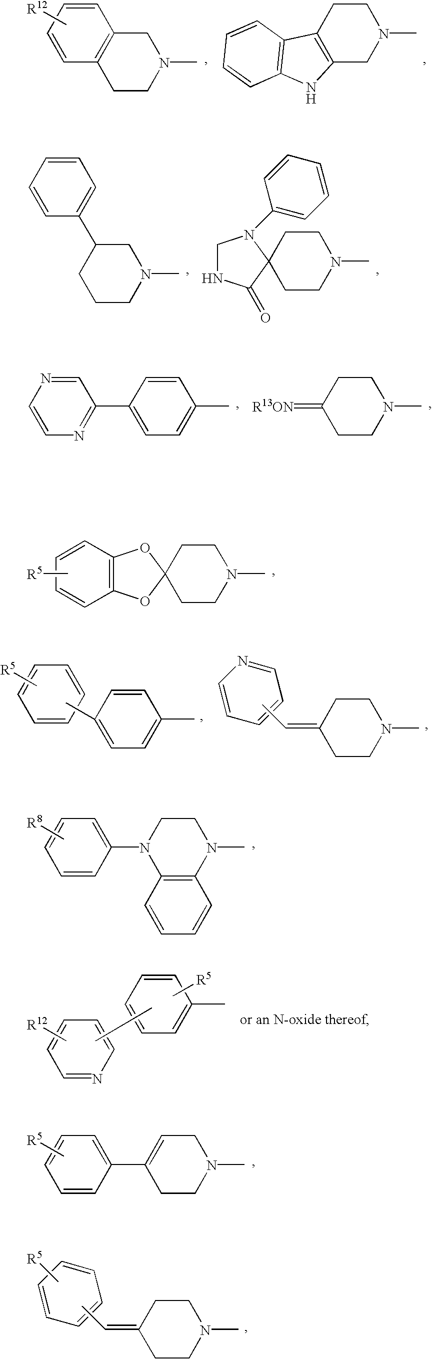

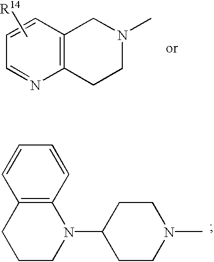

R is R⁴-heteroaryl, R⁵-phenyl, (C₄-C₆)cycloalkenyl, —C(═CH₂)CH₃, —C≡C—CH₃,

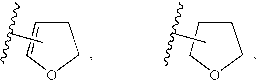

—CH═C(CH₃)₂, or —CH═CH—CH₃;

R² is halo, —W—X, —NH(CH₂)ₘ—W—X, —NHCH(CH₃)—W—X, or

R² is alkyl, alkenyl or —NR¹⁸R¹⁹ which is optionally substituted by —W—X;

R³ is H, halo, alkyl, trifluoromethyl, alkoxy, alkoxyalkyl, hydroxyalkyl, alkylamino, alkylaminoalkyl, dialkylamino, dialkylaminoalkyl, aminoalkyl, aryl, heteroaryl, or CN;

R⁴ is 1 to 3 substituents, which may be the same or different and are independently selected from the group consisting of hydrogen, (C₁-C₆)-alkyl, —CF₃, halogen, —NO₂, —NR¹⁵R¹⁶, (C₁-C₆)alkoxy, (C₁-C₆)alkylthio, (C₁-C₆)alkylsulfinyl, (C₁-C₆)alkylsulfonyl, —COOR¹⁷ or —C(O)NR⁶R⁷;

R⁵ is 1 to 5 substituents, which may be the same or different and are independently selected from the group consisting of hydrogen, halogen, (C₁-C₆)alkyl, hydroxy, (C₁-C₆)alkoxy, —CN, —NH₂, (C₁-C₆)alkylamino, di-((C₁-C₆)alkyl)amino, —CF₃, —OCF₃, —S(O)₀₋₂(C₁-C₆)alkyl and —CH₂—SO₂-phenyl;

R⁶ and R⁷, which may be the same or different, are independently selected from the group consisting of hydrogen and (C₁-C₆)alkyl;

R⁸ is 1 to 5 substituents, which may be the same or different and are independently selected from the group consisting of hydrogen, halogen, (C₁-C₆)alkyl, hydroxy, C₁-C₆ alkoxy, —CN, amino, di-((C₁-C₆)alkyl)amino, —CF₃, —OCF₃, acetyl, —NO₂, hydroxy(C₁-C₆)alkoxy, (C₁-C₆)alkoxyhydroxy, (C₁-C₆)-alkoxy(C₁-C₆)alkoxy, di-((C₁-C₆)-alkoxy)(C₁-C₆)alkoxy, (C₁-C₆)-alkoxy(C₁-C₆)alkoxy-(C₁-C₆)-alkoxy, carboxy(C₁-C₆)-alkoxy, (C₁-C₆)-alkoxycarbonyl(C₁-C₆)alkoxy, (C₃-C₆)cycloalkyl(C₁-C₆)alkoxy, di-((C₁-C₆)alkyl)amino(C₁-C₆)alkoxy, morpholinyl, (C₁-C₆)alkyl-SO₂—, (C₁-C₆)alkyl-SO₂—(C₁-C₆)alkoxy, tetrahydropyranyloxy, (C₁-C₆)alkylcarbonyl(C₁-C₆)-alkoxy, (C₁-C₆)-alkoxycarbonyl, (C₁-C₆)alkylcarbonyloxy(C₁-C₆)-alkoxy, —SO₂NH₂, phenoxy,

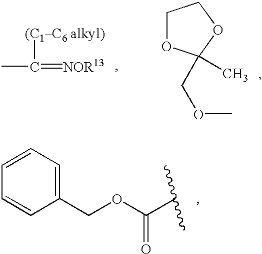

—O—CH₂—P(O)(OR⁶)₂,— and —P(O)(OR⁶)₂; or

adjacent R⁸ substituents together are —O—CH₂—O—, —O—CH₂CH₂—O—, —O—CF₂—O— or —O—CF₂CF₂—O— and form a ring with the carbon atoms to which they are attached;

R⁹ is (C₁-C₆)alkyl, R⁸-phenyl, R⁸-phenyl(C₁-C₆)alkyl, thienyl, pyridyl, (C₃-C₆)-cycloalkyl, (C₁-C₆)alkyl-OC(O)—NH—(C₁-C₆)alkyl-, di-((C₁-C₆)alkyl)aminomethyl, or

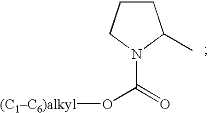

R¹⁰ is 1-2 substituents, which may be the same or different and are independently selected from the group consisting of hydrogen, (C₁-C₆)alkyl, R⁵-aryl and R⁴-heteroaryl, or two R¹⁰ substituents on the same carbon can form ═O;

R¹¹ is hydrogen or (C₁-C₆)alkyl; or R¹⁷ and R¹¹ taken together are —(CH₂)ₚ-A-(CH₂)_{q}, wherein p and q are independently 2 or 3 and A is a bond, —CH₂—, —S— or —O—, and form a ring with the nitrogen to which they are attached;

R¹² is 1-2 substituents, which may be the same or different and are independently selected from the group consisting of hydrogen, (C₁-C₆)alkyl, hydroxy, (C₁-C₆)alkoxy, halogen, and —CF₃;

R¹³ is H, (C₁-C₆)alkyl, phenyl, benzyl, (C₂-C₆)alkenyl, (C₁-C₆)alkoxy(C₁-C₆)alkyl, di-((C₁-C₆)alkyl)amino(C₁-C₆)alkyl, pyrrolidinyl(C₁-C₆)alkyl or piperidino(C₁-C₆)alkyl;

R¹⁴ is H, halogen, (C₁-C₆)alkyl or (C₁-C₆)alkoxy;

R¹⁵ is H or (C₁-C₆)alkyl;

R¹⁶ is H, (C₁-C₆)alkyl-C(O)— or (C₁-C₆)alkyl-SO₂—;

R¹⁷ is (C₁-C₆)alkyl, R⁸-phenyl or R⁸-phenyl(C₁-C₆)alkyl;

R¹⁸ is a bond, —CH₂—, —CH(OH)—, —CH(CH₃)—, or —C(CH₃)₂—; and

R¹⁹ is H or lower alkyl.

U.S. Provisional Application 60/334,385 discloses useful adenosine A₂ₐ receptor antagonist compounds having the structural formula VIII

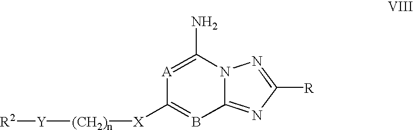

wherein:

A is C(R¹) and B is C(R^{1a}); or A is C(R¹) and B is N; or A is N and B is C(R^{1a}); or A and B are both N;

R¹ and R^{1a} are independently selected from the group consisting of H, (C₁-C₆)-alkyl, halo, CN and —CF₃;

Y is —O—, —S—, —SO—, —SO₂—, R⁵-heteroaryldiyl, R⁵-arylene or

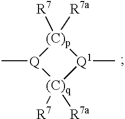

p and q are independently 2-3;

Q and Q¹ are independently selected from the group consisting of

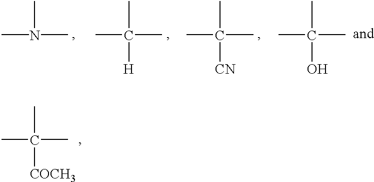

provided that at least one of Q and Q¹ is

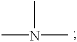

n is 1, 2 or 3; and

(a) A and B are both N, and X is —C(R³)(R^{3a})—, —C(O)—, —O—, —S—, —SO—, —SO₂—, —N(R⁹)—, R⁴-arylene or R⁴-heteroaryldiyl; or A and B are both N, Y is a bond and X is —C(O)—, R⁴-arylene or R⁴-heteroaryldiyl; or

(b) A is C(R¹), B is N, and X is —C(R³)(R^{3a})—, —C(O)—, —O—, —S—, —SO—, —SO₂—, —N(R⁹)—, R⁴-arylene or R⁴-heteroaryldiyl; or A is C(R¹), B is N, Y is a bond, and X is —C(O)— or R⁴-heteroaryldiyl; or

(c) A is C(R¹), B is C(R^{1a}), and X is —C(R³)(R^{3a})—, —C(O)—, —O—, —S—, —SO—, —SO₂—, R⁴-arylene, R⁴-heteroaryldiyl, or —N(R⁹)—, provided that when X is —N(R⁹)—, R²—Y is not aryl(C₁-C₆alkyl)arylene; or A is C(R¹), B is C(R^{1a}), Y is a bond, and X is —C(R³)(R^{3a})—, —C(O)—, —O—, —S—, —SO—, —SO₂—, R⁴-arylene, —N(R⁹)— or R⁴-heteroaryldiyl, provided that when X is —N(R⁹)— or R⁴-heteroaryldiyl, R² is not phenyl or phenyl(C₁-C₆)alkyl;

or n is 2 or 3; and

(d) A is N, B is C(R^{1a}), and X is —C(R³)(R^{3a})—, —C(O)—, —O—, —S—, —SO—, —SO₂—, —N(R⁹)—, R⁴-arylene or R⁴-heteroaryldiyl; or A is N, B is C(R^{1a}), Y is a bond and X is —C(O)—, —N(R⁹)—, R⁴-arylene or R⁴-heteroaryldiyl;

R is R⁵⁻aryl, R⁵⁻heteroaryl, R⁶—(C₂-C₆)alkenyl or R⁶-(C₂-C₆)alkynyl;

R² is R⁵⁻aryl, R⁵⁻heteroaryl, R⁵⁻aryl(C₁-C₆)alkyl or R⁵⁻heteroaryl(C₁-C₆)alkyl; or R²—Y is selected from the group consisting of

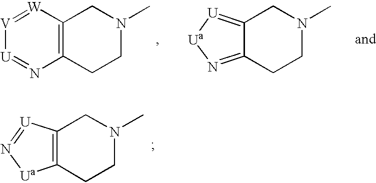

U, V, and W are independently selected from the group consisting of N and CR¹, provided that at least one of U, V and W is CR¹;

U^{a} is —O—, —S—, —NH—, or —N(C₁-C₆ alkyl)-;

R³ and R^{3a} are independently selected from the group consisting of H, —OH, C₁-C₆ alkyl, hydroxy(C₁-C₆)alkyl, (C₁-C₆)alkoxy(C₁-C₆)alkyl, amino(C₁-C₆)alkyl, (C₁-C₆)alkylamino(C₁-C₆)alkyl and di(C₁-C₆)alkylamino(C₁-C₆)alkyl;

R⁴ is 1-3 substituents selected from the group consisting of H, (C₁-C₆)alkyl, —OH, (C₁-C₆)alkoxy, (C₁-C₆)alkoxy(C₁-C₆)alkoxy, halo, —CF₃, and —CN;

R⁵ is 1-3 substituents independently selected from the group consisting of H, (C₁-C₆)alkyl, —OH, (C₁-C₆)alkoxy, (C₁-C₆)alkoxy(C₁-C₆)alkyl, (C₁-C₆)alkoxy(C₁-C₆)-alkoxy, halo, —CF₃, —CN, —NH₂, (C₁-C₆)alkylamino, di(C₁-C₆)alkylamino, amino(C₁-C₆)-alkyl, (C₁-C₆)alkylamino(C₁-C₆)alkyl, di(C₁-C₆)alkylamino(C₁-C₆)alkyl, (C₁-C₆)alkanoylamino, (C₁-C₆)alkanesulfonylamino, (C₁-C₆)alkylthio, (C₁-C₆)alkylthio(C₁-C₆)alkyl, R⁶-(C₂-C₆)alkenyl and R⁶-(C₂-C₆)alkynyl;

R⁶ is 1 to 3 substituents independently selected from the group consisting of H, (C₁-C₆)alkyl, —OH, (C₁-C₆)alkoxy and halo;

R⁷ and R^{7a} are independently selected from the group consisting of H, (C₁-C₆)alkyl, (C₁-C₆)alkoxy(C_{1-C}₆)alkyl, R⁸-aryl and R⁸-heteroaryl, or an R⁷ and an R^{7a} substituent on the same carbon can form ═O;

R⁸ is 1 to 3 substituents independently selected from H, (C₁-C₆)alkyl, —OH, (C₁-C₆)alkoxy, (C₁-C₆)alkoxy(C₁-C₆)alkoxy, halo, —CF₃, and —CN; and

R⁹ is H, C₁-C₆ alkyl, hydroxy(C₂-C₆)alkyl, (C₁-C₆)alkoxy(C₂-C₆)alkyl, amino(C₂-C₆)alkyl, (C₁-C₆)alkylamino(C₂-C₆)alkyl and di(C₁-C₆)alkylamino(C₂-C₆)alkyl.

Preferred compounds of formula VIII are those wherein A is N or C(R¹) and B is C(R^{1a}), with compounds wherein A is N and B is C(R^{1a}) being more preferred. Another group of preferred compounds is that wherein X is —O—, —S—, —N(R⁹)— or R⁴-arylene. Preferred definitions for Y are a bond or piperazinyl (i.e., a group of the formula

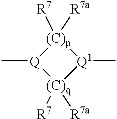

wherein Q and Q¹ are each nitrogen, p and q are each 2, and each R⁷ and each R^{7a} is H). R² is preferably R⁵-aryl. R is preferably furyl.

Preferred specific examples of compounds of formula VIII include compounds of the formula

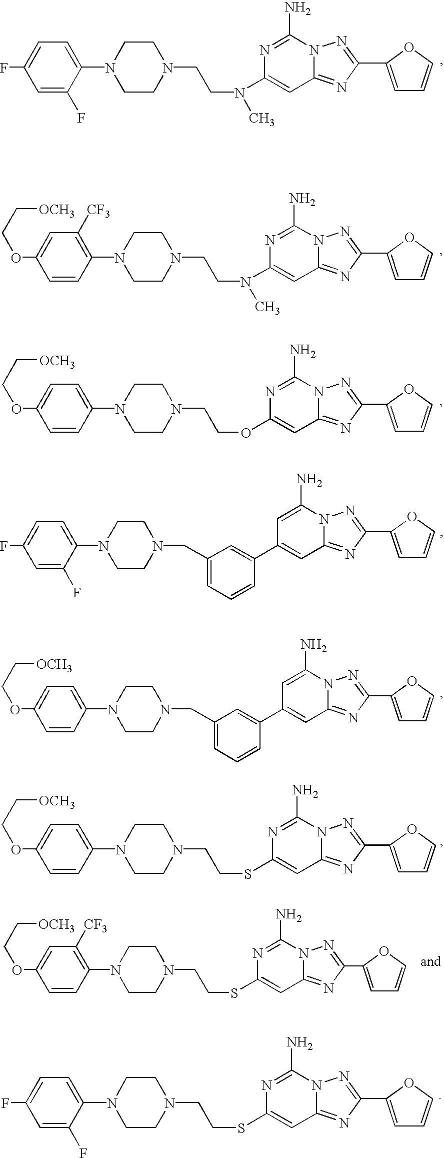

WO 01/02409 discloses useful adenosine A₂ₐ receptor antagonist compounds having the structural formula IX

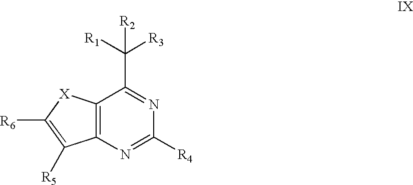

wherein

X is O or S;

R₁ and R₂ are independently selected from hydrogen, alkyl, aryl, hydroxy, alkoky, aryloxy, cyano, nitro, CO₂R₇, COR₇, OCOR₇, CONR₇R₈, CONR₇NR₈R₉, OCONR₇R₈, NR₇R₈, NR₇COR₈, NR₇CONR₈R₉, NR₇CO₂R₈, NR₇SO₂R₈, NR₇CONR₈NR₉R₁₀, NR₇NR₈CO₂R₉, NR₇NR₈CONR₉R₁₀, NR₇SO₂NR₈R₉, SO₂R₇,SOR₇, SR₇ and SO₂NR₇R₈, or R₁ and R₂ together form a carbonyl group (C═O), an oxime group (C═NOR₁₁), an imine group (C═NR₁₁) or a hydrazine group (C═NNR₁₁R₁₂), or R₁ and R₂ together form a 5, 6 or 7 membered carbocyclic or heterocyclic ring;

R₃ is alkyl or aryl;

R₄, R₅ and R₆ ate independently selected from hydrogen, alkyl, aryl, halogen, hydroxy, nitro, cyano, alkoxy, aryloxy, CO₂R₇, COR₇, OCOR₇, SO₂R₇, SOR₇, SR₇, SO₂NR₇R₈,, CONR₇R₈, CONR₇NR₈R₉, OCONR₇R₈, NR₇R₈, NR₇COR₈, NR₇CONR₈R₉, NR₇CO₂R₈, NR₇SO₂R₈, CR₇═NOR₈, NR₇CONR₈NR₉R₁₀, NR₇NR₈CO₂R₉, NR₇NR₈CONR₉R₁₀, SO₂NR₇NR₈R₉, NR₇SO₂NR₈R₉, NR₇NR₈SO₂R₉, NR₇NR₈CO₂R₉, NR₇NR₈R₉ and NR₇CSNR₈R₉, or R₅ and R₆ together form a 5, 6 or 7 membered carbocyclic or heterocyclic ring; and

R₇, R₈, R₉, R₁₀, R₁₁ and R₁₂ are independently selected from hydrogen, alkyl and aryl, or a pharmaceutically acceptable salt or prodrug thereof.

Agents known to be useful in the treatment of depression (“antidepressants”) which can be administered in combination with an adenosine A₂ₐ receptor antagonist include: selective serotonin reuptake inhibitors such as fluoxetine, sertraline, paroxetine, citalopram, mirtazepine and fluvoxamine; selective norepinephrine reuptake inhibitors such as reboxetine, desipramine, amitriptyline, nortriptyline and imipramine; mixed serotonin/ norepinephrine reuptake inhibitors such as venlafaxine, buproprion, nefazodone and milnacipran, and combinations thereof.

Agents known to be useful in the treatment of anxiety-related disorders (i.e., anxiolytics) which can be administered in combination with an adenosine A₂ₐ receptor antagonist include alprazolam, buspirone, lorazepam, diazepam, clonazepam, doxepin, chlordiazepoxide and meprobamate, and combinations thereof.

The US patents and applications cited herein are incorporated herein by reference. The adenosine A₂ₐ receptor antagonists are prepared by known methods as described in the cited patents and applications. The antidepressants and anxiolytics are commercially available and are described in the literature, e.g., in The Physicians' Desk Reference (Montvale: Medical Economics Co., Inc., 2001)

It is contemplated that two or more A₂ₐ receptor antagonists could be administered in combination with one or more antidepressants or one or more anxiolytics to treat depression or anxiety-related disorders; it is also contemplated that one or more antidepressants and one or more anxiolytics could be combined with one or more A₂ₐ receptor antagonists for the treatment of depression or anxiety-related disorders.

The pharmacological activity of the compounds of the invention was determined by the following in vitro and in vivo assays to measure A₂ₐ receptor activity.

Human Adenosine A₂ₐ and A₁ Receptor Competition Binding Assay Protocol

Membrane sources:

A₂ₐ: Human A₂ₐ Adenosine Receptor membranes, Catalog #RB-HA2a, Receptor Biology, Inc., Beltsville, Md. Dilute to 17 μg/100 μl in membrane dilution buffer (see below).

Assay Buffers:

Membrane dilution buffer: Dulbecco's Phosphate Buffered Saline (Gibco/BRL)+10 mM MgCl₂.

Compound Dilution Buffer: Dulbecco's Phosphate Buffered Saline (Gibco/BRL)+10 mM MgCl₂ supplemented with 1.6 mg/ml methyl cellulose and 16% DMSO. Prepared fresh daily.

Ligands:

A₂ₐ: [3H]-SCH 58261, custom synthesis, AmershamPharmacia Biotech, Piscataway, N.J. Stock is prepared at 1 nM in membrane dilution buffer. Final assay concentration is 0.5 nM.

A₁: [3H]-DPCPX, AmershamPharmacia Biotech, Piscataway, N.J. Stock is prepared at 2 nM in membrane dilution buffer. Final assay concentration is 1 nM.

Non-specific Binding:

A₂ₐ: To determine non-specific binding, add 100 nM CGS 15923 (RBI, Natick, Mass.). Working stock is prepared at 400 nM in compound dilution buffer.

A₁: To determine non-specific binding, add 100 μM NECA (RBI, Natick, Mass.). Working stock is prepared at 400 μM in compound dilution buffer.

Compound Dilution:

Prepare 1 mM stock solutions of compounds in 100% DMSO. Dilute in compound dilution buffer. Test at 10 concentrations ranging from 3 μM to 30 pM. Prepare working solutions at 4×final concentration in compound dilution buffer.

Assay procedure:

Perform assays in deep well 96 well plates. Total assay volume is 200 μl. Add 50 μl compound dilution buffer (total ligand binding) or 50 μl CGS 15923 working solution (A₂ₐ non-specific binding) or 50 μl NECA working solution (A₁ non-specific binding) or 50 μl of drug working solution. Add 50 μl ligand stock ([3H]-SCH 58261 for A₂ₐ, [3H]-DPCPX for A₁). Add 100 μl of diluted membranes containing the appropriate receptor. Mix. Incubate at room temperature for 90 minutes. Harvest using a Brandel cell harvester onto Packard GF/B filter plates. Add 45 μl Microscint 20 (Packard), and count using the Packard TopCount Microscintillation Counter. Determine IC₅₀ values by fitting the displacement curves using an iterative curve fitting program (Excel). Determine Ki values using the Cheng-Prusoff equation.

Haloperidol-induced Catalepsy in the Rat

Male Sprague-Dawley rats (Charles River, Calco, Italy) weighing 175-200 g are used. The cataleptic state is induced by the subcutaneous administration of the dopamine receptor antagonist haloperidol (1 mg/kg, sc), 90 min before testing the animals on the vertical grid test. For this test, the rats are placed on the wire mesh cover of a 25×43 plexiglass cage placed at an angle of about 70 degrees with the bench table. The rat is placed on the grid with all four legs abducted and extended (“frog posture”). The use of such an unnatural posture is essential for the specificity of this test for catalepsy. The time span from placement of the paws until the first complete removal of one paw (decent latency) is measured maximally for 120 sec.

The selective A_{2A} adenosine antagonists under evaluation are administered orally at doses ranging between 0.03 and 3 mg/kg, 1 and 4 h before scoring the animals.

In separate experiments, the anticataleptic effects of the reference compound, L-DOPA (25, 50 and 100 mg/kg, ip), were determined.

6-OHDA Lesion of the Middle Forebrain Bundle in Rats

Adult male Sprague-Dowley rats (Charles River, Calco, Como, Italy), weighing 275-300 g, are used in all experiments. The rats are housed in groups of 4 per cage, with free access to food and water, under controlled temperature and 12 hour light/dark cycle. The day before the surgery the rats are fasted over night with water ad libitum.

Unilateral 6-hydroxydopamine (6-OHDA) lesion of the middle forebrain bundle is performed according to the method described in Ungerstedt et al, Brian Research, 24 (1970), p. 485-493, and Ungerstedt, Eur. J. Pharmacol., 5 (1968), p.107-110, with minor changes. Briefly, the animals are anaesthetized with chloral hydrate (400 mg/kg, ip) and treated with desipramine (10 mpk, ip) 30 min prior to 6-OHDA injection in order to block the uptake of the toxin by the noradrenergic terminals. Then, the animals are placed in a stereotaxic frame. The skin over the skull is reflected and the stereotaxic coordinates (−2.2 posterior from bregma (AP), +1.5 lateral from bregma (ML), 7.8 ventral from dura (DV) are taken, according to the atlas of Pellegrino et al (Pellegrino L. J., Pellegrino A. S. and Cushman A. J., A Stereotaxic Atlas of the Rat Brain. 1979, New York: Plenum Press). A burr hole is then placed in the skull over the lesion site and a needle, attached to a Hamilton syringe, is lowered into the left MFB. Then 8 μg 6-OHDA-HCl is dissolved in 4 μl of saline with 0.05% ascorbic acid as antioxidant, and infused at the constant flow rate of 1 μl/1 min using an infusion pump. The needle is withdrawn after additional 5 min and the surgical wound is closed and the animals left to recover for 2 weeks.

Two weeks after the lesion the rats are administered with L-DOPA (50 mg/kg, ip) plus Benserazide (25 mg/kg, ip) and selected on the basis of the number of full contralateral turns quantified in the 2 h testing period by automated rotameters (priming test). Any rat not showing at least 200 complete turns/2 h is not included in the study.

Selected rats receive the test drug 3 days after the priming test (maximal dopamine receptor supersensitivity). The new A_{2A} receptor antagonists are administered orally at dose levels ranging between 0.1 and 3 mg/kg at different time points (i.e., 1, 6, 12 h) before the injection of a subthreshold dose of L-DOPA (4 mpk, ip) plus benserazide (4 mpk, ip) and the evaluation of turning behavior.

In the binding assay, adenosine A₂ₐ receptor antagonists for use in the present invention preferably show A₂ₐ Ki vaules of 0.3 to 100 nM, with preferred compounds showing Ki values between 0.3 and 5.0 nM.

Selectivity is determined by dividing Ki for A, receptor by Ki for A₂ₐ receptor. Preferred compounds of the invention have a selectivity ranging from about 100 to about 2000.

Preferred compounds showed a 50-75% decrease in descent latency when tested orally at 1 mg/kg for anti-cataleptic activity in rats.

In the 6-OHDA lesion test, rats dosed orally with 1 mg/kg of the preferred compounds performed 170-440 turns in the two-hour assay period.

In the haloperidol-induced catalepsy test, a combination of sub-threshold amount of a compound of formula I and a sub-threshold amount of L-DOPA showed a significant inhibition of the catalepsy, indicating a synergistic effect. In the 6-OHDA lesion test, test animals administered a combination of a compound of formula I and a sub-threshold amount of L-DOPA demonstrated significantly higher (6-fold) contralateral turning.

Depression and anxiety are measure by the following tests, wherein immobility is an indication of depression.

Mouse Tail Suspension Test

The tail suspension test is based on the observation that a mouse suspended by the tail shows alternate periods of agitation and immobility. The mouse, acoustically and visually isolated, is hung on the hook by an adhesive tape placed 20 mm from the extremity of its tail and it is kept 150 mm away from the nearest object. The duration of immobility is recorded for 6 min. The sum of immobility periods (duration of immobility) is measured by an observer who was unaware of the drug treatments. Each mouse is used only once for each experimental session.

Mouse and Rat Forced Swim Test

Mouse: In the forced swimming test, mice are dropped individually into glass cylinders (height: 25 cm, diameter: 10 cm) containing 10 cm water, maintained at 25° C., and left there for 6 min. A mouse is judged to be immobile when it floats in an upright position, and makes only small movements to keep its head above water. The duration of immobility is recorded during the last 4-min of the 6-min testing period. The sum of immobility periods (duration of immobility) is measured by an observer who is unaware of the drug treatments. Each mouse is used only once for each experimental session.

Rat: Rat is placed in a cylinder 40 cm high and 18 cm in diameter containing 20 cm of water at 25° C. The animal is left to swim in the water for 15 min before being removed, allowed to dry beside a heated enclosure and returned to its home cage. Twenty-four h later, the animal is exposed again to the conditions outlined above and the total immobility time during a 5-min period recorded (test session). Furthermore, we also measure the active behavior of the animal as the time spent in climbing. Drugs are given 3 times before testing: 24, 5 and 1 h. In each test the measurements are always made under blind conditions.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 70 percent active ingredient. Suitable solid carriers are known in the art, e.g. magnesium carbonate, magnesium stearate, talc, sugar, lactose. Tablets, powders, cachets and capsules can be used as solid dosage forms suitable for oral administration.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby solidify.

Liquid form preparations include solutions, suspensions and emulsions. As an example may be mentioned water or water-propylene glycol solutions for parenteral injection.

Liquid form preparations may also include solutions for intranasal administration.

Aerosol preparations suitable for inhalation may include solutions and solids in powder form, which may be in combination with a pharmaceutically acceptable carrier, such as an inert compressed gas.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

Preferably the compounds are administered orally.

Preferably, when the combination of drugs is administered in a single pharmaceutical composition, the pharmaceutical preparation is in unit dosage form. In such form, the preparation is subdivided into unit doses containing appropriate quantities of each active component, e.g., an amount effective to achieve the desired purpose.

The amount and frequency of administration of the compounds in the combination of this invention will be regulated according to the judgment of the attending clinician considering such factors as age, condition and size of the patient as well as severity of the symptoms being treated. Determination of the proper dosage for a particular situation is within the skill of the art. Generally, treatment is initiated with smaller dosages which are less than the optimum dose of the compound. Thereafter, the dosage is increased by small increments until the optimum effect under the circumstances is reached. For convenience, the total daily dosage may be divided and administered in portions during the day, if desired.

The combination of drugs can be administered individually, either simultaneously or sequentially, in any conventional dosage form such as capsule, tablet, powder, cachet, suspension, solution, suppository, nasal spray, etc. Different drugs can be administered in different dosage forms. When used in combination, the dosage levels of the individual components are preferably lower than the recommended individual dosages because of the advantageous effect of the combination.

The quantity of adenosine A₂ₐ receptor antagonist in a unit dose of preparation may be varied or adjusted from about 0.1 mg to 1000 mg, more preferably from about 1 mg to 300 mg, according to the particular application. A typical recommended dosage regimen for an adenosine A₂ₐ receptor antagonist is oral administration of from 10 mg to 2000 mg/day, preferably 10 to 1000 mg/day, in two to four divided doses to treat depression or anxiety-related disorders.

The doses and dosage regimens of the antidepressant and anxiolytic components of the combination will be determined by the attending clinician in view of the approved doses and dosage regimen known in the art, e.g., in the package insert or other published information, taking into consideration the age, sex and condition of the patient and the severity of the disease.

When the adenosine A₂ₐ receptor antagonist and antidepressant or anxiolytic are to be administered separately, they can be provided in a kit comprising in a single package, one container comprising an adenosine A₂ₐ receptor antagonist in a pharmaceutically acceptable carrier, and a separate container comprising an antidepressant or anxiolytic in a pharmaceutically acceptable carrier, with the adenosine A₂ₐ receptor antagonist and the antidepressant or anxiolytic agent being present in an amount such that the combination is effective to treat depression or anxiety-related disorders. A kit is advantageous for administering a combination when, for example, the components must be administered at different time intervals or when they are in different dosage forms (i.e., tablet and capsule).

The following are examples of pharmaceutical dosage forms suitable for the present invention. Those skilled in the art will recognize that dosage forms are suitable for single actives (i.e. “Active” is an A₂ₐ receptor antagonist or an antidepressant or an anxiolytic), or can contain both components (ie, “Active” comprises both an adenosine A₂ₐ receptor antagonist and an antidepressant or anxiolytic). The scope of the invention in its pharmaceutical composition aspect is not to be limited by the examples provided.

### Pharmaceutical Dosage Form Examples

### EXAMPLE A

### Tablets

| No. | Ingredients | mg/tablet | mg/tablet |
|---|---|---|---|
| 1. | Active compound | 100 | 500 |
| 2. | Lactose USP | 122 | 113 |
| 3. | Corn Starch, Food Grade, as a | 30 | 40 |
| | 10% paste in Purified Water | | |
| 4. | Corn Starch, Food Grade | 45 | 40 |
| 5. | Magnesium Stearate | 3 | 7 |
| | Total | 300 | 700 |

Method of Manufacture

Mix Item Nos. 1 and 2 in a suitable mixer for 10-15 minutes. Granulate the mixture with Item No. 3. Mill the damp granules through a coarse screen (e.g., ¼″, 0.63 cm) if necessary. Dry the damp granules. Screen the dried granules if necessary and mix with Item No. 4 and mix for 10-15 minutes. Add Item No. 5 and mix for 1-3 minutes. Compress the mixture to appropriate size and weigh on a suitable tablet machine.

### EXAMPLE B

### Capsules

| No. | Ingredient | mg/capsule | mg/capsule |
|---|---|---|---|
| 1. | Active compound | 100 | 500 |
| 2. | Lactose USP | 106 | 123 |
| 3. | Corn Starch, Food Grade | 40 | 70 |
| 4. | Magnesium Stearate NF | 7 | 7 |
| | Total | 253 | 700 |

Method of Manufacture

Mix Item Nos. 1, 2 and 3 in a suitable blender for 10-15 minutes. Add Item No. 4 and mix for 1-3 minutes. Fill the mixture into suitable two-piece hard gelatin capsules on a suitable encapsulating machine.

While the present invention has been described in conjunction with the specific embodiments set forth above, many alternatives, modifications and variations thereof will be apparent to those of ordinary skill in the art. All such alternatives, modifications and variations are intended to fall within the spirit and scope of the present invention.

We claim:

1. A method of treating depression or anxiety-related disorders comprising administering to a mammal in need of such treatment an effective amount of a combination of an adenosine A_{2A} antagonist and an antidepressant or an anxiolytic.

2. The method of claim 1 wherein the adenosine A₂ₐ receptor antagonist is selected from those described in formulas I, II, III, IVA, IVB, V, VI, VII, VIII and IX as disclosed in the specification.

3. The method of claim 1 wherein the antidepressant is selected from selective serotonin reuptake inhibitors, selective norepinephrine reuptake inhibitors, and mixed serotonin/norepinephrine reuptake inhibitors.

4. The method of claim 3 wherein the antidepressant is selected from fluoxetine, sertraline, paroxetine, citalopram, mirtazepine, fluvoxamine, reboxetine, desipramine, amitriptyline, nortriptyline, imipramine, venlafaxine, buproprion, nefazodone and milnacipran.

5. The method of claim 1 wherein the anxiolytic is selected from alprazolam, buspirone, lorazepam, diazepam, clonazepam, doxepin, chlordiazepoxide and meprobamate.

6. The method of claim 1 wherein the adenosine A₂ₐ receptor antagonist is selected from those described in formulas I, II, III, IVA, IVB, V, VI, VII, VIII and IX as disclosed in the specification; the antidepressant is selected from fluoxetine, sertraline, paroxetine, citalopram, mirtazapine, fluvoxamine, reboxetine, desipramine, amitriptyline, nortriptyline, imipramine, venlafaxine, buproprion, nefazodone and milnacipran; and the anxiolytic is selected from alprazolam, buspirone, lorazepam, diazepam, clonazepam, doxepin, chlordiazepoxide and meprobamate.

7. The method of claim 6 wherein the adenosine A₂ₐ receptor antagonist is represented by the structural formula I 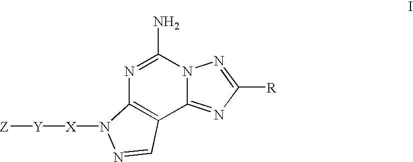
or a pharmaceutically acceptable salt thereof, wherein
R is R¹-furanyl, R¹-thienyl, R¹-pyridyl, R¹-pyridyl N-oxide, R¹-oxazolyl, R¹⁰-phenyl, R¹-pyrrolyl or C₄-C₆ cycloalkenyl;
X is C₂-C₆ alkylene or —C(O)CH₂—;
Y is —N(R²)CH₂CH₂N(R³)—, —OCH₂CH₂N(R²)—, —O—, —S—, —CH₂S—, —(CH₂)₂—NH—, or 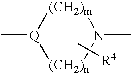
and
Z is R⁵-phenyl, R⁵-phenyl(C₁-C₆)alkyl, R⁵-heteroaryl, diphenylmethyl, R⁶—C(O)—, R⁶—SO₂—, R⁶—OC(O)—, R⁷—N(R⁸)—C(O)—, R⁷—N(R⁸)—C(S)—, 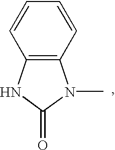
phenyl-CH(OH)—, or phenyl-C(═NOR²)—; or when Q is 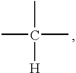
Z is also phenylamino or pyridylamino; or
Z and Y together are 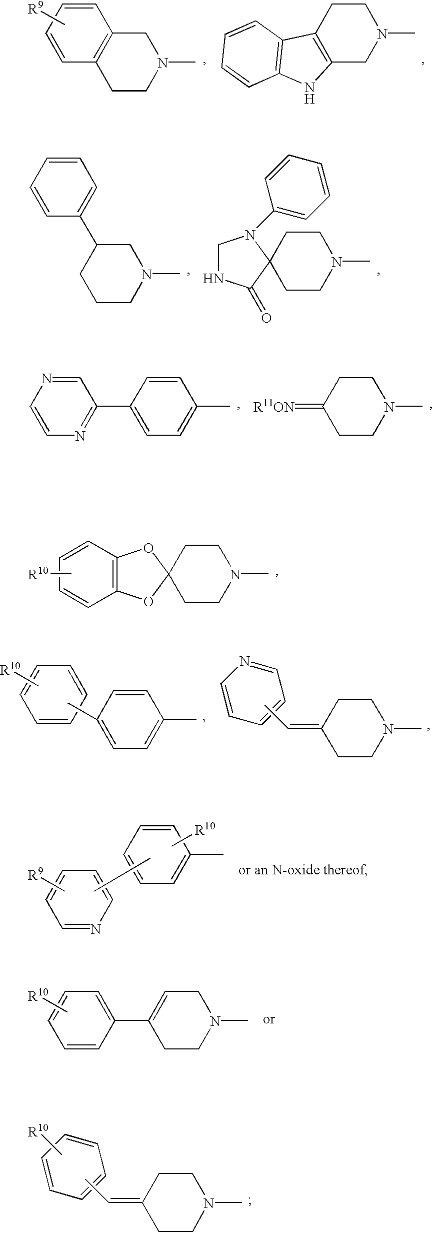
R¹ is 1 to 3 substituents independently selected from hydrogen, C₁-C₆-alkyl, —CF₃, halogen, —NO₂, —NR¹²R¹³, C₁-C₆ alkoxy, C₁-C₆ alkylthio, C₁-C₆ alkylsulfinyl, a C₁-C₆ alkylsulfonyl;
R² and R³ are independently selected from the group consisting of hydrogen and C₁-C₆ alkyl;
m and n are independently 2-3;
Q is 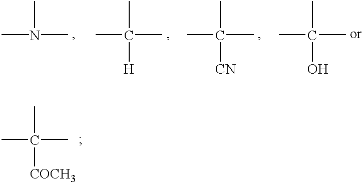
R⁴ is 1-2 substituents independently selected from the group consisting of hydrogen and C₁-C₆alkyl, or two R⁴ substituents on the same carbon can form ═O;
R⁵ is 1 to 5 substituents independently selected from the group consisting of hydrogen, halogen, C₁-C₆ alkyl, hydroxy, C₁-C₆ alkoxy, —CN, di-((C₁-C₆)alkyl)amino, —CF₃, —OCF₃, acetyl, —NO₂, hydroxy(C₁-C₆)alkoxy, (C₁-C₆)-alkoxy(C₁-C₆)alkoxy, di-((C₁-C₆)-alkoxy)(C₁-C₆)alkoxy, (C₁-C₆)-alkoxy(C₁-C₆)alkoxy-(C₁-C₆)-alkoxy, carboxy(C₁-C₆)-alkoxy, (C₁-C₆)-alkoxycarbonyl(C₁-C₆)alkoxy, (C₃-C₆)cycloalkyl(C₁-C₆)alkoxy, di-((C₁-C₆)alkyl)amino(C₁-C₆)alkoxy, morpholinyl, (C₁-C₆)alkyl-SO₂—, (C₁-C₆)alkyl-SO—(C₁-C₆)alkoxy, tetrahydropyranyloxy, (C₁-C₆)alkylcarbonyl(C₁-C₆)-alkoxy, (C₁-C₆)-alkoxycarbonyl, (C₁-C₆)alkylcarbonyloxy(C₁-C₆)-alkoxy, —SO₂NH₂, phenoxy, 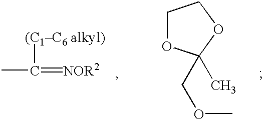
or adjacent R⁵ substituents together are —O—CH₂—O—, —O—CH₂CH₂—O—, —O—CF₂—O— or —O—CF₂CF₂—O— and form a ring with the carbon atoms to which they are attached;
R⁶ is (C₁-C₆)alkyl, R⁵-phenyl, R⁵-phenyl(C₁-C₆)alkyl, thienyl, pyridyl, (C₃-C₆)-cycloalkyl, (C₁-C₆)alkyl-OC(O)—NH—(C₁-C₆)alkyl-, di-((C₁-C₆)alkyl)aminomethyl, or 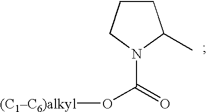
R⁷ is (C₁-C₆)alkyl, R⁵-phenyl or R⁵-phenyl(C₁-C₆)alkyl;
R⁸ is hydrogen or C₁-C₆ alkyl; or R⁷ and R⁸ together are —(CH₂)ₚ-A-(CH₂)_{q}, wherein p and q are independently 2 or 3 and A is a bond, —CH₂—, —S— or —O—, and form a ring with the nitrogen to which they are attached;
R⁹ is 1-2 groups independently selected from hydrogen, C₁-C₆ alkyl, hydroxy, C₁-C₆ alkoxy, halogen, —CF₃ and (C₁-C₆)alkoxy(C₁-C₆)alkoxy;
R¹⁰ is 1 to 5 substituents independently selected from the group consisting of hydrogen, halogen, C₁-C₆ alkyl, hydroxy, C₁-C₆ alkoxy, —CN, —NH₂, C₁-C₆alkylamino, di-((C₁-C₆)alkyl)amino, —CF₃, —OCF₃ and —S(O)₀₋₂(C₁-C₆)alkyl;
R¹¹ is H, C₁-C₆ alkyl, phenyl, benzyl, C₂-C₆ alkenyl, C₁-C₆ alkoxy(C₁-C₆)alkyl, di-((C₁-C₆)alkyl)amino(C₁-C₆)alkyl, pyrrolidinyl(C₁-C₆)alkyl or piperidino(C₁-C₆)alkyl;
R¹² is H or C₁-C₆ alkyl; and
R¹³ is (C₁-C₆)alkyl-C(O)— or (C₁-C₆)alkyl-SO₂—.

8. The method of claim 7 wherein the adenosine A₂ₐ receptor antagonist is represented by the formula 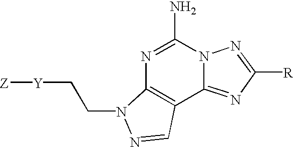
wherein R and Z-Y are as defined in the following table:

| Z—Y— | R |
|---|---|
| 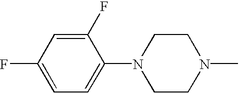 | 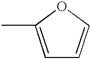 |
| 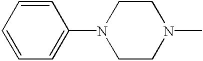 | 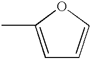 |
| 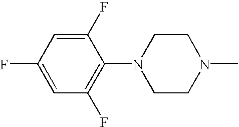 | 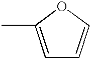 |
| 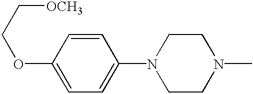 | 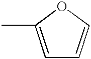 |
| 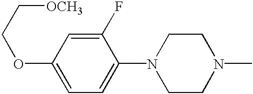 | 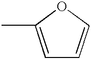 |
| 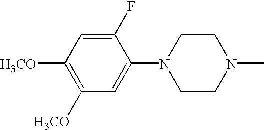 | 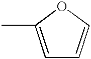 |
| 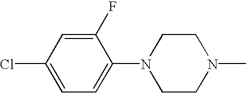 | 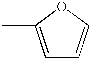 |
| 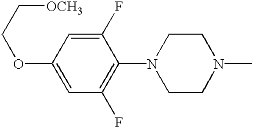 | 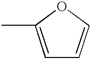 |
| 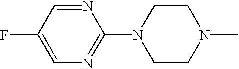 | 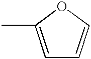 |
| 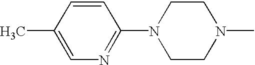 | 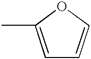 |
| 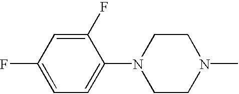 | 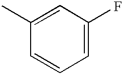 |
| 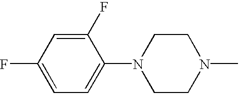 | 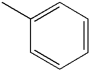 |

9. A pharmaceutical composition comprising a therapeutically effective amount of a combination of an adenosine A₂ₐ receptor antagonist and an antidepressant or an anxiolytic in a pharmaceutically acceptable carrier.

10. The composition of claim 9 wherein the adenosine A₂ₐ receptor antagonist is selected from those described in formulas I, II, III, IVA, IVB, V, VI, VII, VIII and IX as disclosed in the specification.

11. The composition of claim 9 wherein the antidepressant is selected from selective serotonin reuptake inhibitors, selective norepinephrine reuptake inhibitors, and mixed serotonin/norepinephrine reuptake inhibitors.

12. The composition of claim 11 wherein the antidepressant is selected from fluoxetine, sertraline, paroxetine, citalopram, mirtazapine, fluvoxamine, reboxetine, desipramine, amitriptyline, nortriptyline, imipramine, venlaflaxine, buproprion, nefazodone and milnacipran.

13. The composition of claim 9 wherein the anxiolytic is selected from alprazolam, buspirone, lorazepam, diazepam, clonazepam, doxepin, chlordiazepoxide and meprobamate.

14. The composition of claim 9 wherein the adenosine A₂ₐ receptor antagonist is selected from those described in formulas I, II, III, IVA, IVB, V, VI, VII, VIII and IX as disclosed in the specification; the antidepressant is selected from fluoxetine, sertraline, paroxetine, citalopram, mirtazapine, fluvoxamine, reboxetine, desipramine, amitriptyline, nortriptyline, imipramine, venlaflaxine, buproprion, nefazodone and milnacipran; and the anxiolytic is selected from alprazolam, buspirone, lorazepam, diazepam, clonazepam, doxepin, chlordiazepoxide and meprobamate.

15. The composition of claim 14 wherein the adenosine A₂ₐ receptor antagonist is represented by the structural formula I 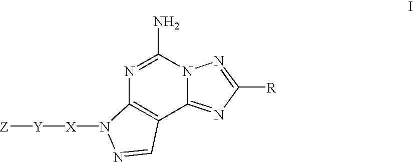
or a pharmaceutically acceptable salt thereof, wherein
R is R¹-furanyl, R¹-thienyl, R¹-pyridyl, R¹-pyridyl N-oxide, R¹-oxazolyl, R¹⁰-phenyl, R¹-pyrrolyl or C₄-C₆ cycloalkenyl;
X is C₂-C₆ alkylene or —C(O)CH₂—;
Y is —N(R²)CH₂CH₂N(R³)—, —OCH₂CH₂N(R²)—, —O—, —S—, —CH₂S—, —(CH₂)₂—NH—, or 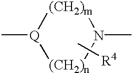
and
Z is R⁵-phenyl, R⁵-phenyl(C₁-C₆)alkyl, R⁵-heteroaryl, diphenylmethyl, R⁶—C(O)—, R⁶—SO₂—, R⁶—OC(O)—, R⁷—N(R⁸)—C(O)—, R⁷—N(R⁸)—C(S)—, 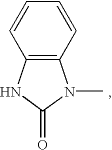
phenyl-CH(OH)—, or phenyl-C(═NOR²)—; or when Q is 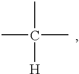
Z is also phenylamino or pyridylamino; or
Z and Y together are 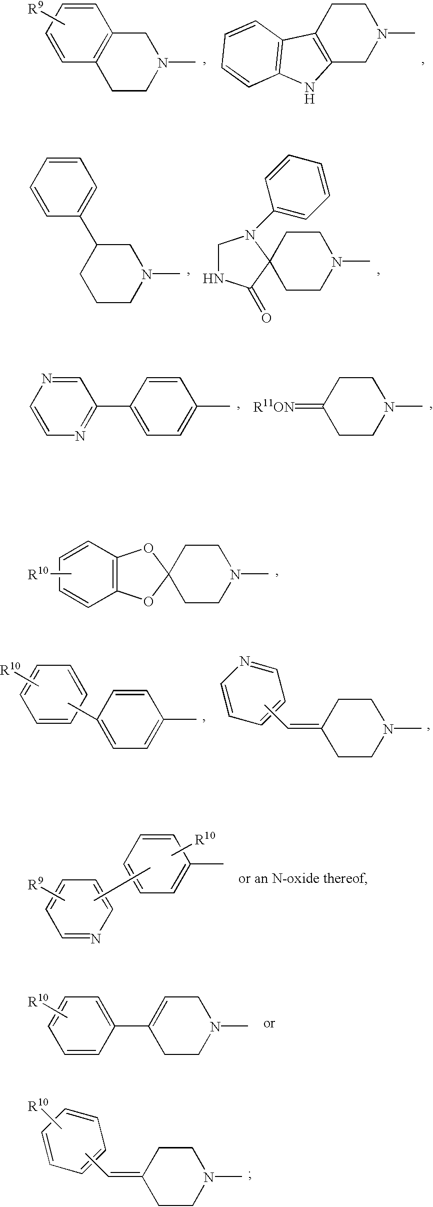
R¹ is 1 to 3 substituents independently selected from hydrogen, C₁-C₆-alkyl, —CF₃, halogen, —NO₂, —NR¹²R¹³, C₁-C₆ alkoxy, C₁-C₆ alkylthio, C₁-C₆ alkylsulfinyl, and C₁-C₆ alkylsulfonyl;
R² and R³ are independently selected from the group consisting of hydrogen and C₁-C₆ alkyl;
m and n are independently 2-3;
Q is 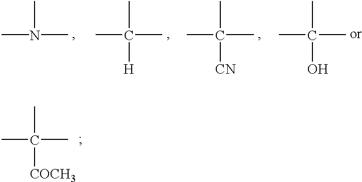
R⁴ is 1-2 substituents independently selected from the group consisting of hydrogen and C₁-C₆alkyl, or two R⁴ substituents on the same carbon can form ═O;
R⁵ is 1 to 5 substituents independently selected from the group consisting of hydrogen, halogen, C₁-C₆ alkyl, hydroxy, C₁-C₆ alkoxy, —CN, di-((C₁-C₆)alkyl)amino, —CF₃, —OCF₃, acetyl, —NO₂, hydroxy(C₁-C₆)alkoxy, (C₁-C₆)-alkoxy(C₁-C₆)alkoxy, di-((C₁-C₆)-alkoxy)(C₁-C₆)alkoxy, (C₁-C₆)-alkoxy(C₁-C₆)alkoxy-(C₁-C₆)-alkoxy, carboxy(C₁-C₆)-alkoxy, (C₁-C₆)-alkoxycarbonyl(C₁-C₆)alkoxy, (C₃-C₆)cycloalkyl(C₁-C₆)alkoxy, di-((C₁-C₆)alkyl)amino(C₁-C₆)alkoxy, morpholinyl, (C₁-C₆)alkyl-SO₂—, (C₁-C₆)-SO—(C₁-C₆)alkoxy, tetrahydropyranyloxy, (C₁-C₆)alkylcarbonyl(C₁-C₆)-alkoxy, (C₁-C₆)-alkoxycarbonyl, (C₁-C₆)alkylcarbonyloxy(C₁-C₆)-alkoxy, —SO₂NH₂, phenoxy, 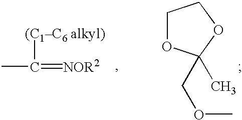
or adjacent R⁵ substituents together are —O—CH₂—O—, —O—CH₂CH₂—O—, —O—CF₂—O— or —O—CF₂CF₂—O— and form a ring with the carbon atoms to which they are attached;
R⁶ is (C₁-C₆)alkyl, R⁵-phenyl, R⁵-phenyl(C₁-C₆)alkyl, thienyl, pyridyl, (C₃-C₆)-cycloalkyl, (C₁-C₆)alkyl-OC(O)—NH—(C₁-C₆)alkyl-, di-((C₁-C₆)alkyl)aminomethyl, or 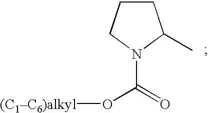
R⁷ is (C₁-C₆)alkyl, R⁵-phenyl or R⁵-phenyl(C₁-C₆)alkyl;
R⁸ is hydrogen or C₁-C₆ alkyl; or R⁷ and R⁸ together are —(CH₂)ₚ-A-(CH₂)_{q}, wherein p and q are independently 2 or 3 and A is a bond, —CH₂—, —S— or —O—, and form a ring with the nitrogen to which they are attached;
R⁹ is 1-2 groups independently selected from hydrogen, C₁-C₆ alkyl, hydroxy, C₁-C₆ alkoxy, halogen, —CF₃ and (C₁-C₆)alkoxy(C₁-C₆)alkoxy;
R¹⁰ is 1 to 5 substituents independently selected from the group consisting of hydrogen, halogen, C₁-C₆ alkyl, hydroxy, C₁-C₆ alkoxy, —CN, —NH₂, C₁-C₆alkylamino, di-((C₁-C₆)alkyl)amino, —CF₃, —OCF₃ and —S(O)₀₋₂(C₁-C₆)alkyl;
R¹¹ is H, C₁-C₆ alkyl, phenyl, benzyl, C₂-C₆ alkenyl, C₁-C₆ alkoxy(C₁-C₆)alkyl, di-((C₁-C₆)alkyl)amino(C₁-C₆)alkyl, pyrrolidinyl(C₁-C₆)alkyl or piperidino(C₁-C₆)alkyl;
R¹² is H or C₁-C₆ alkyl; and
R¹³ is (C₁-C₆)alkyl-C(O)— or (C₁-C₆)alkyl-SO₂—.

16. The composition of claim 15 wherein the adenosine A₂ₐ receptor antagonist is represented by the formula 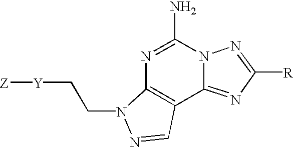
wherein R and Z-Y are as defined in the following table:

| Z—Y— | R |
|---|---|
| 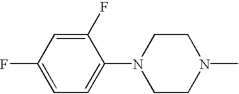 | 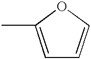 |
| 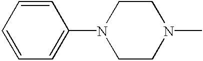 | 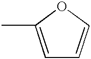 |
| 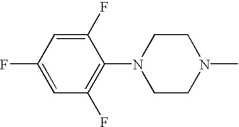 | 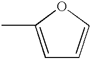 |
| 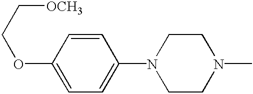 | 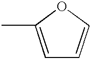 |
| 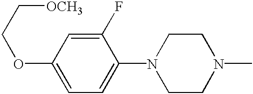 | 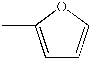 |
| 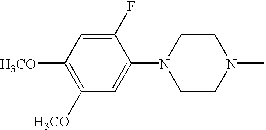 | 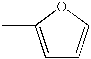 |
| 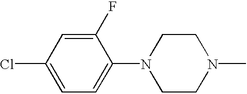 | 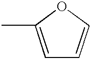 |
| 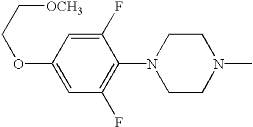 | 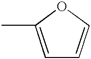 |
| 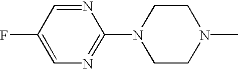 | 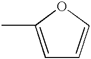 |
| 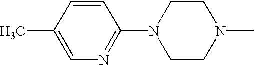 | 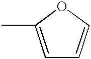 |
| 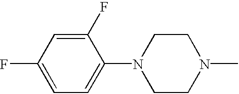 | 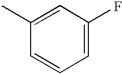 |
| 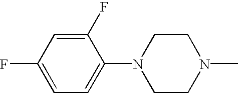 | 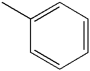 |

17. A kit comprising in a single package, one container comprising an adenosine A₂ₐ receptor antagonist in a pharmaceutically acceptable carrier, and a separate container comprising an antidepressant in pharmaceutically acceptable carrier or an anxiolytic in a pharmaceutically acceptable carrier, with the adenosine A₂ₐ receptor antagonist and the antidepressant or anxiolytic agent being present in amounts such that the combination is effective to treat depression or anxiety-related disorders.
